(19) United States
(12) Patent Application Publication
(10) Pub. No.: US 2011/0207832 A1
(43) Pub. Date: Aug. 25, 2011

(54) SKIN DISINFECTANT COMPOSITION AND METHODS FOR MANUFACTURING AND USING

(75) Inventors: James A. Roszell, Las Vegas, NV (US); Jie Zhang, Henderson, NV (US)

(73) Assignee: Skinvisible Pharmaceuticals, Inc., Las Vegas, NV (US)

(21) Appl. No.: 13/030,719

(22) Filed: Feb. 18, 2011

Related U.S. Application Data
(60) Provisional application No. 61/306,424, filed on Feb. 19, 2010.

(51) Int. Cl. A61K 47/32; A61P 31/02

(52) U.S. Cl. 514/772.6

(57) ABSTRACT

A disinfectant active ingredient containing composition is provided. The composition includes a disinfectant active ingredient, a hydrophobic polymer/hydrophilic polymer adduct comprising a poly(vinylpyrrolidone/alkylene) copolymer wherein the alkylene group contains at least 10 carbon atoms and a poly(vinylpyrrolidone imidizole) copolymer, and at least about 50 wt. % water. The disinfectant active ingredient containing composition can include a compatiblizing amount of a long chain organic acid having a carbon chain of at least 8 carbon atoms to help compatibize the disinfectant active ingredient with the hydrophobic polymer/hydrophilic polymer adduct. Methods of using and manufacturing the disinfectant active ingredient containing composition are provided.

### CROSS-REFERENCE TO RELATED APPLICATION

The present application includes the disclosure of U.S. Application Ser. No. 61/306,424 that was filed with the United States Patent and Trademark Office on Feb. 19, 2010. A priority right is claimed to U.S. Provisional Application Ser. No. 61/306,424, to the extent appropriate. The completed disclosure of U.S. Provisional Application Ser. No. 61/306,424 is incorporated herein by reference.

### FIELD OF THE INVENTION

The invention relates to a skin disinfectant composition, to a method for using a skin disinfectant composition, and to a method for manufacturing a skin disinfectant composition.

### BACKGROUND

Skin bonding polymer compositions are described that include a hydrophobic polymer/hydrophilic polymer adduct and other components. These compositions can be provided as topical compositions to be applied to skin tissue. Exemplary skin bonding polymer compositions are described in U.S. Pat. No. 6,756,059, assigned to Skinvisible Pharmaceuticals, Inc. In addition, see U.S. Patent Publication No. 2002/0051795. Because of the highly hydrophobic nature of certain hydrophobic polymer/hydrophilic polymer adducts described in U.S. Pat. No. 6,756,059, ionic and water-soluble active ingredients may have difficulty being absorbed into or contained within the hydrophobic polymer/hydrophilic polymer adduct. Because a large number of active ingredients are salts, it is desirable to provide a composition that absorbs or binds ionic and water-soluble active ingredients and allows the active ingredients to be released, over time, to skin tissue.

Chlorhexidine gluconate is an antiseptic used as an active ingredient in dental and non-dental applications. Dental products that contain chlorhexidine gluconate are available under the names PERIDEX, PERIOCHIP, and PERIOGARD ORAL RINSE. A non-dental product that includes chlorhexidine gluconate is available under the name HIBICLENS from Regent Medical. The HIBICLENS product is available for general skin cleansing as a surgical scrub, and as a pre-operative skin preparation. The HIBICLENS product is aqueous and contains 4.0 wt. % chlorhexidine gluconate and 4.0 wt. % isopropanol.

Chlorhexidine digluconate is available in a teat dip for use in the dairy industry to prevent mastitis. An exemplary teat dip product contains 0.5 wt. % chlorhexidine digluconate.

There is a general desire to provide products that reduce irritation to skin, chapping and redness of skin, and that provide disinfectant properties over a relatively long period of time.

### SUMMARY

A disinfectant active ingredient containing composition is provided. The composition includes a disinfectant active ingredient, a hydrophobic polymer/hydrophilic polymer adduct comprising a poly(vinylpyrrolidone/alkylene) copolymer wherein the alkylene group contains at least 10 carbon atoms and a poly(vinylpyrrolidone imidizole) copolymer, and at least about 50 wt. % water. The disinfectant active ingredient containing composition can include a compatiblizing amount of a long chain organic acid having a carbon chain of at least 8 carbon atoms to help compatiblize the disinfectant active ingredient with the hydrophobic polymer/hydrophilic polymer adduct.

A method of using a disinfectant active ingredient containing composition is provided that includes a step of applying the disinfectant active ingredient containing composition to skin tissue. The step of applying can include rubbing the composition onto skin tissue by hand application.

A method for manufacturing a disinfectant active ingredient containing composition is provided. The method includes a step of forming a polymer adduct comprising poly(vinylpyrrolidone-alkylene) copolymer wherein the alkylene group contains at least 10 carbon atoms, and a poly(vinylpyrrolidone-imidizole) copolymer, and forming an emulsion therefrom.

### DETAILED DESCRIPTION

A skin disinfectant composition is provided that exhibits prolonged disinfectant properties. The skin disinfectant composition can be referred to as the disinfectant composition or more simply as the composition. In general, the disinfectant properties can include antimicrobial activity, antibacterial activity, antiviral activity, antifungal activity, or a mixture thereof. Prolonged disinfectant properties refers to disinfectant properties that persist over a period of time. In general, the persistence can be considered sufficient so that the skin tissue having the skin disinfectant composition applied thereto can exhibit disinfectant properties for at least one hour after application of the composition to the skin tissue (preferably for at least two hours) and taking into account washing the skin tissue every half hour for 30 seconds with a mild soap. The disinfectant composition preferably provides disinfectant properties at least about four hours after application to skin tissue and taking into account washing the skin tissue every 30 minutes for 30 seconds with a mild soap. The disinfectant composition can be characterized as a composition that resists washing from skin tissue using mild soap.

The skin disinfectant composition can be applied to skin tissue on virtually any part of the body to provide disinfectant properties. One area of the body often in need of disinfectant is the hands. When the skin disinfectant composition is available for application to the hands to provide disinfectant properties, the skin disinfectant composition can be referred to as a hand disinfectant composition.

The skin disinfectant composition can be provided in the form of a lotion and applied to skin tissue by rubbing the composition onto the skin tissue. The skin disinfectant composition can have a viscosity that allows it to be applied to skin tissue conveniently as a lotion. The skin disinfectant composition can have a viscosity that is sufficiently high so that the lotion can be applied from a container (e.g., a tube or a bottle) to a person's hand or a location on the person's body, and the lotion can be rubbed onto the skin tissue. When provided as a lotion, the hand disinfectant composition can have a viscosity of greater than about 3,000 cSt (centistokes) under normal conditions (room temperature and atmospheric pressure). When provided as a lotion, the skin disinfectant composition can be referred to as a skin disinfectant lotion or more simply as a lotion. The skin disinfectant composition can be provided in a form having a viscosity of less than about 3,000 cSt. When the skin disinfectant composition is provided having a viscosity of less than about 3,000 cSt, the skin disinfectant composition can be called a skin disinfectant liquid or more simply as a liquid. In addition, the skin disinfectant composition can be applied in the form of a gel.

The skin disinfectant composition includes an antimicrobial component, a skin bonding polymer component, and water. Additional components that can be included in the skin disinfecting composition include surfactant, pH modifying agent, buffering agent, coloring agent, preservative, thickening agent, emollient, humectant, antioxidant, fragrance, and chelating agent. The skin disinfecting composition can include any one or more of these additional components.

The skin disinfectant composition can be provided as an emulsion. Exemplary types of emulsions include oil in water emulsions, and water in oil in water emulsions.

The skin disinfectant composition can be provided so that the skin disinfectant composition exhibits at least a 2 log reduction Gram negative bacteria in 15 minutes. Preferrably, the skin disinfectant composition can be provided so that it exhibits at least a 3 log reduction against Gram negative bacteria in 15 minutes.

### Polymer Component

The skin disinfectant composition can include a polymer component. The polymer component can be provided as a component that exhibits a tendency to bond to skin tissue, and hold the disinfectant active ingredient in place and allow the disinfectant active ingredient to be released at a desired rate. When the polymer component is provided as a polymer that has a tendency to bond to skin tissues, the polymer component can be characterized as a skin bonding polymer component.

The polymer component can include a hydrophobic polymer/hydrophilic polymer adduct and can include other components. The polymer component of the composition can be, at least in part, responsible for holding or isolating the disinfectant active ingredient and releasing the disinfectant active ingredient at a controlled rate. The polymer component can help hold the disinfectant active ingredient in proximity to the skin tissue once it is applied to skin tissue. By binding to skin tissue and holding on to the disinfectant active ingredient, the polymer component can help deliver the disinfectant active ingredient to the skin tissue to provide a desired level of activity for a desired length of time. For example, the disinfectant active ingredient containing composition can be provided so that it adheres or binds to skin tissue for at least about one hour, and preferably at least about two hours, and holds the disinfectant active ingredient in proximity to the skin tissue for that length of time.

The polymer component can be prepared by mixing a hydrophobic polymer and a hydrophilic polymer, with the application heat, to provide an interaction between the hydrophobic polymer and the hydrophilic polymer. It should be understood that the reference to mixing, with the application of heat, means that the hydrophobic polymer and the hydrophilic polymer are in a fluid (i.e., liquid) state so that they sufficiently mix. When the polymers are sufficiently mixed, it is believed that an interaction forms between the hydrophobic polymer and the hydrophilic polymer. The mixing temperature can be at least about 50° C., at least about 60° C., or at least about 70° C. to generate this interaction. In addition, the mixing temperature can be at least about 80° C. or at least about 90° C., but should not be so high that it causes degradation of the polymers. Once the polymers have been mixed sufficiently to provide an interaction between polymers, the polymers are allowed to cool to room temperature. In theory, it is understood that heating allows the polymers to “uncoil” so that the hydrophobic polymers and the hydrophilic polymers can interact and form a level of alignment, and the cooling allows the polymers to coil together in their aligned arrangement. As a result, the hydrophobic polymer and the hydrophilic polymers retain their interaction as a result of coiling. It is theorized that this type of interaction can be considered a type of “annealing” where the heating of the polymers allows them to align and the subsequent cooling allows the aligned polymers to coil and thereby retain their alignment. It is observed that simply mixing the hydrophobic polymer and the hydrophilic polymers without heating and cooling does not create the same level of interaction between the hydrophobic and the hydrophilic polymers. In any event, the interaction is additionally understood to be a type of “complex” formation.

It is theorized that the interaction exhibited between the hydrophobic polymer and the hydrophilic polymer is a type of complex formation reaction, and that the complex, once formed, can be stable in water at temperatures up to 65° C. and at a pH range of 3.0 to 9.0. By stable, it is meant that the complex does not favor disassociation under these conditions, and tends to exhibit a desired shelf life. It is believed that this interaction provides the composition with an ability to become emulsified in water, exhibit a desired shelf life, bind to skin tissue, and hold the disinfectant active component. The result of the interaction between the hydrophobic polymer component and the hydrophilic polymer component can be referred to as a hydrophobic polymer/hydrophilic polymer adduct. The term “adduct” is used to refer to the interaction between the hydrophobic polymer component and the hydrophilic polymer component. The interaction may be a form of complexing. The adduct, however, can be referred to as a complex because it is understood to be a complex. Accordingly, the term “adduct” is not meant to limit the polymer component to a particular theory of interaction. The interaction, however, provides an emulsion containing the adduct with an enhanced shelf life compared with an emulsion where an adduct has not been formed. An emulsion containing the adduct can exhibit enhanced resistance to splitting into separate phases. An emulsion formed by simply mixing the hydrophobic polymer and the hydrophilic polymer under conditions so that the adduct is not formed results in a composition that has a greater tendency to split into separate phases.

The hydrophobic polymer includes at least one hydrophobic polymer and can include a mixture of hydrophobic polymers. The hydrophobic polymer can include components having repeating pyrrolidone/alkylene groups. Exemplary polymers having repeating pyrrolidone/alkylene groups include poly(vinylpyrrolidone/alkylene) polymers. Poly(vinylpyrrolidone/alkylene) polymers include those polymers obtained by polymerizing alkylene substituted vinylpyrrolidone. Poly(vinylpyrrolidone/alkylene) polymers can be represented by the following general formula:

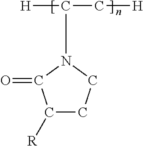

wherein R represents a carbon chain such as an alkylene group and n represents the number of repeating units. The R group is preferably sufficiently long so that the polymer remains relatively water insoluble and should not be too long so that the polymer is difficult to melt process. The alkylene group can contain at least about 10 carbon atoms so that the poly(vinylpyrrolidone/alkylene) polymer exhibits hydrophobic properties. If the alkylene group is too long, it may be difficult to process the poly(vinylpyrrolidone/alkylene) polymer. In general, the alkylene group can be provided having up to about 34 carbon atoms. The alkylene group can contain at least about 10 carbon atoms to about 32 carbon atoms. The alkylene group can contain about 14 carbon atoms to about 30 carbon atoms. The polymer can be referred to as a copolymer.

The poly(vinylpyrrolidone/alkylene) polymers can have a molecular weight that is sufficiently high so that the polymer maintains its water insolubility but the molecular weight should not be so high that it becomes difficult to melt process the polymer. The weight average molecular weight of the poly(vinylpyrrolidone/alkylene) polymer can be between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of about 6,000 to about 30,000, the poly(vinylpyrrolidone/alkylene) polymer can have about 20 to about 80 repeating units, and can have about 30 to about 50 repeating units. It should be understood that repeating units refer to the residues of vinylpyrrolidone/alkylene groups.

Exemplary poly(vinylpyrrolidone/alkylene) polymers include poly(vinylpyrrolidone/1-eicosene), poly(vinylpyrrolidone/hexadecene), and poly(vinylpyrrolidone/tricontanyl). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity. Poly(vinylpyrrolidone/tricontanyl) can be referred to as PVPT.

PVPE and PVPH differ in the length of the hydrocarbon side chain, and are used extensively in the skin care industry, usually at concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy. PVPT generally has a waxy consistency.

The alkylene group of PVPE has about 20 carbon atoms, the alkylene group of PVPH has about 16 carbon atoms, and the alkylene group of PVPT has about 30 carbon atoms.

The hydrophobic polymer can be provided as a single poly(vinylpyrrolidone/alkylene) polymer or as a mixture of different poly(vinylpyrrolidone/alkylene) polymers. When provided as a mixture of different poly(vinylpyrrolidone/alkylene) polymers, the mixture can be provided as a mixture where one PVP polymer (or mixture) has an alkylene group of about 10 to about 24 carbon atoms, and the other PVP polymer (or mixture) has an alkylene group of about 26 to about 34 carbon atoms. The mixture can be provided at a weight ratio of the PVP polymer having an alkylene group of about 10 to about 24 carbon atoms to the PVP polymer having an alkylene group of about 26 to about 34 carbon atoms of about 10:1 to about 2:1, or about 8:1 to about 4:1. Furthermore, the PVP polymer having an alkylene group of about 10 carbon atoms to about 24 carbon atoms can be provided as a mixture of PVP polymers. For example, the mixture can be provided as a PVP polymer (or mixture) having an alkylene group of about 10 carbon atoms to about 18 carbon atoms, and a PVP polymer (or mixture) having an alkylene group of about 18 carbon atoms to about 24 carbon atoms. When provided as a mixture, the weight ratio of the PVP polymer having an alkylene group of about 10 carbon atoms to about 18 carbon atoms to the PVP polymer having an alkylene group of about 18 carbon atoms to about 24 carbon atoms, can be provided at about 10:1 to about 2:1, or can be provided at about 8:1 to about 4:1.

The hydrophilic polymer can include at least one hydrophilic polymer and can include a mixture of hydrophilic polymers. The hydrophilic polymer can have a molecular weight sufficient to allow the hydrophilic polymer to mix and interact with the hydrophobic polymer composition to form an adduct or complex.

The hydrophilic polymer can be provided as a polyvinylpyrrolidone polymer having a hydrophilic substituent. The hydrophobic polymer described above can be characterized as a polyvinylpyrrolidone polymer having a hydrophobic substituent. Because both the hydrophobic polymer and the hydrophilic polymer are based upon polyvinylpyrrolidone polymers, the Applicants have found enhanced interaction therebetween. It is theorized that the polyvinylpyrrolidone groups are groups that interact or complex with each other. In the case of the polyvinylpyrrolidone polymer having a hydrophilic substituent, the hydrophilic substituent can be provided as an imidizole ring. When the hydrophilic substituent is an imidizole ring, the hydrophilic polymer can be referred to as a polyvinylpyrrolidone imidizole copolymer. In general, the imidizole ring provides a positive charge for the copolymer. An exemplary polyvinylpyrrolidone imidizole copolymer is polyvinylpyrrolidone/vinylimidizolemethylsulfonate copolymer. An exemplary form of this polymer is available under the name Luviquat from BASF and can be referred to as Polyquaternium 44. In general, Polyquaternium 44 is a copolymer based on 80 wt. % polyvinylpyrrolidone and 20 wt. % vinylimidizolemethylsulfonate, and the polymer has an overall cationic charge. The monomeric copolymer structure of polyquaternium 44 is provided as:

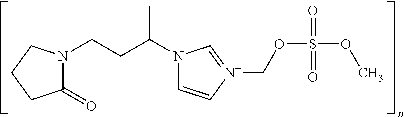

In the above structure, n represents the number of repeating units, and the value can vary depending upon the molecular weight of the polymer. By way of example, the value of n can be about 20 to about 1000. The weight average molecular weight of the Polyquaternium 44 should be sufficiently large so that the polymer, once complexed with the hydrophobic polymer composition, can provide a stable emulsion. In addition, the Polyquaternium 44 should not be too large so that it is difficult to handle because of its high viscosity. In general, the weight average molecular weight can be about 25,000 to about 2,000,000, and can be about 50,000 to about 1,000,000.

The hydrophobic polymer and the hydrophilic polymer can be combined and heated to at least about 50° C. The composition can be heated to at least about 60° C., or at least about 70° C. under mixing to form a complex between the hydrophobic and hydrophilic polymers. The resulting complex or adduct can be allowed to cool to room temperature.

The hydrophilic polymer composition can be provided as a solution in water. In the case of Luviquat from BASF, the polymer composition is available as a 20% solution meaning that the composition contains 80 wt. % water and 20 wt. % polymer.

The hydrophobic polymer and the hydrophilic polymer can be mixed together in amounts that provide a desirable, stable product when subsequently formed into an emulsion. The ratio of the hydrophobic polymer composition to the hydrophilic polymer composition can be referred to as a weight ratio of the polymer components, and the weight ratio does not include the amount of water that may be present in the hydrophilic polymer composition. Furthermore, the weight ratio does take into account the potential existence of mixtures of polymers that can be used to form either or both of the hydrophobic polymer and the hydrophilic polymer. In general, the weight ratio of the hydrophobic polymer composition to the hydrophilic polymer composition can be about 6:1 to about 1:1, about 5:1 to about 1.5:1, and about 4:1 to about 2:1.

For a preferred polymer, the polymer adduct can be prepared by mixing 64 wt % Polyquaternium 44 (20% solution), 25 wt. % polyvinylpyrrolidone/hexadecene, 5% polyvinylpyrrolidone/eicosene, 5 wt. % polyvinylpyrrolidone/tricontanyl, and 1 wt. % preservative with sufficient heat to allow the polymers to interact (for example, at least 50° C.). The resulting polymer adduct (hydrophobic polymer/hydrophilic polymer adduct) can be characterized as about a 50% solution. The polymer adduct can be used in the formulation of a skin disinfectant composition containing chlorhexidine.

Additional components can be added to the hydrophobic polymer/hydrophilic polymer adduct. For example, it may be desirable to add a component that helps stabilize the hydrophobic polymer/hydrophilic polymer adduct, and to help preserve and/or maintain the composition.

The disinfectant active ingredient containing composition can include the polymer component or polymer adduct in an amount sufficient to provide desired bonding properties of the composition. For example, the disinfectant active ingredient containing composition can include at least about 2 wt. % of the polymer component or polymer adduct. It should be understood that the reference to the amount of the polymer component or polymer adduct refers to the amount of polymer and does not include the amount of water that may be present therein. In addition, the composition can contain at least 3 wt. % of the polymer component or polymer adduct, and preferably at least about 4 wt. % of the polymer component or polymer adduct. In addition, the disinfectant active ingredient containing composition can include a sufficient amount of the polymer component or polymer adduct to desirably protect the disinfectant active ingredient from degradation and allow the disinfectant active ingredient containing composition to deliver the disinfectant active ingredient to skin tissue upon application of the disinfectant active ingredient containing composition to the skin tissue. The disinfectant active ingredient containing composition can contain the polymer component in an amount of less than about 20 wt. %, and preferably in an amount of less than about 15 wt. %. Exemplary ranges of polymer component include about 2 wt. % to about 20 wt. %, about 4 wt. % to about 15 wt. %, and about 5 wt. % to about 8 wt. %.

### Disinfectant Active Component

The skin disinfectant composition includes a disinfectant active component. The skin disinfectant composition can include any disinfectant active component that is compatible with the skin bonding polymer component and provides desired disinfectant properties. In general, compatibility of the disinfectant active component with the skin bonding polymer component refers to the lack of phase separation between the disinfectant active component and the skin bonding polymer component. It is generally desirable for the skin bonding polymer component to hold the disinfectant active component to skin tissue to provide the desired disinfectant properties for a desired length of time.

Chlorhexidine can be referred to as 1,6-di(4-chlorophenyl-diguanido) hexane. In general, chlorhexidine is a strong base and is practically insoluble in water (0.008% wt/vol. at 20° C.). To make chlorhexidine soluble in water, it is typically reacted with acids to form salts of the RX₂ type. See Denton, Disinfection, Sterilization and Preservation, Fifth Edition, Chapter 15, pages 321-336, Lippincott, Williams and Wilkins, Dec. 15, 2000. It should be understood that the reference to “% wt/vol. at 20° C.” refers to the weight percent of the component that is soluble in 100 ml water at 20° C.

Chlorhexidine digluconate is a commonly used antiseptic. Because of its relatively high water solubility, chlorhexidine digluconate has a tendency to phase separate from the skin bonding polymer component. Accordingly, chlorhexidine can be modified to make it more compatible with the skin bonding polymer component. In general, the modification can provide a modified chlorhexidine that is more compatible with the skin bonding polymer component so that the skin bonding polymer component holds the modified chlorhexidine to skin tissue. The chlorhexidine modified to be more compatible with the skin bonding polymer component can be referred to as modified chlorhexidine. The modified chlorhexidine can be provided as a chlorhexidine salt.

Chlorhexidine can be provided as a chlorhexidine salt having a water solubility of less than about 1% wt/vol in water at 20° C. by selecting the acid that reacts with the chlorhexidine to provide a more neutral chlorhexidine salt having the desired level of low water solubility. In general, it is desirable to neutralize the chlorhexidine so that the chlorhexidine does not harm the skin tissue. Furthermore, it is desirable to provide the chlorhexidine salt as a relatively water insoluble salt so that the skin bonding polymer component can hold the chlorhexidine salt while the polymer component is bonded to the skin tissue. Because the polymer component can hold the chlorhexidine salt while the polymer component is bonded to the skin tissue, the chlorhexidine salt can be made available to provide desired disinfectant properties while the polymer component remains bonded to the skin tissue. In addition, a portion of the chlorhexidine salt can be provided as relatively water soluble, and this portion is generally available to provide disinfectant properties once the composition is applied to skin tissue

Exemplary acids that can react with chlorhexidine to provide a chlorhexidine salt having a relatively low water solubility include fatty acids, polycarboxylic acids, mineral acids, and mixtures thereof. Exemplary fatty acids include those fatty acids having a fatty chain that helps reduce water solubility. In general, the fatty chain can have at least about eight carbon atoms. The fatty chain should not be so large that the fatty acid is not capable of sufficiently reacting with the chlorhexidine. For example, it may be desirable to provide the fatty chain having less than about 30 carbon atoms. Furthermore, the fatty chain can be characterized as saturated or unsaturated. Exemplary fatty acids that can be used include stearic acid, oleic acid, and mixtures of stearic acid and oleic acid. The polycarboxylic acid can be used to provide cross linking between chlorhexidine molecules to decrease water solubility. Cross linking can create an increased molecular weight that can provide reduced water solubility. Exemplary polycarboxylic acids include dicarboxylic acids. The polycarboxylic acids, such as dicarboxylic acids, can be characterized as aliphatic, aromatic, or cyclic. In order to provide decreased water solubility, the polycarboxylic acids can be preferably provided so that they do not internally form a salt with the chlorhexidine by forming a cyclic molecule. For example, it may be desirable to avoid having a dicarboxylic acid react with both ends of the chlorhexidine molecule to form a cyclic salt. Accordingly, the number of carbon atoms between carboxylic acid groups can be about 1 to about 12. An exemplary dicarboxylic acid includes succinic acid. Mineral acids can be provided to react with the chlorhexidine to reduce water solubility. Exemplary mineral acids include hydrochloric acid, hydrobromic acid, nitric acid, sulfuric acid, carbonic acid, phosphoric acid, or mixtures thereof.

The chlorhexidine salt that can be used include those that can be characterized as relatively water insoluble. In general, a relatively water insoluble chlorhexidine salt can be characterized as a salt having a water solubility of less that about 1% wt/vol. in water at 20° C., preferably less that about 0.5% wt/vol. in water at 20° C., and more preferably less than about 0.1% wt/vol. in water at 20° C. In addition, the chlorhexidine salt that can be used include those that can be characterized as relatively water soluble. By providing chlorhexidine salt that is relatively water soluble, the relatively water soluble chlorhexidine salt can be available immediately after application to skin tissue to provide disinfectant properties. Preferably, the chlorhexidine salt includes chlorhexidine salt that is available immediately upon application to skin tissue to provide disinfectant properties, and chlorhexidine salt that remains with the polymer adduct to provide prolonged or sustained disinfectant properties. The chlorhexidine salt can be provided as a mixture of chlorhexidine salts.

The chlorhexidine salt can be prepared by mixing chlorhexidine with an acid in water at a temperature sufficient to enhance the salt forming reaction. The temperature of the composition can be heated to about 45° C. to about 50° C. to enhance the salt formation. In general, as the chlorhexidine salt forms, it tends to precipitate out and form a slurry. Although basic chlorhexidine can be provided as a starting material, it should be appreciated that other salts of chlorhexidine can be provided as a starting material. For example, chlorhexidine gluconate can be used as a starting material.

Exemplary forms of chlorhexidine salt include stearic acid salts of chlorhexidine (chlorhexidine stearate), succinate acid salts of chlorhexidine (chlorhexidine succinate), and mixtures of chlorhexidine stearate and chlorhexidine succinate. A desired release of chlorhexidine from the skin disinfectant composition can be found when a mixture of chlorhexidine stearate and chlorhexidine succinate is used. When a mixture of chlorhexidine stearate and chlorhexidine succinate is used, the mixture can be provided at a weight ratio of chlorhexidine stearate to chlorhexidine succinate of about 1:10 to about 10:1, and preferably 1:5 to about 5:1.

The skin disinfectant composition can include disinfectant active component in an amount sufficient to provide desired disinfectant properties. Exemplary ranges of the disinfectant active component in the skin disinfectant composition include about 0.1 wt. % to about 5 wt. %, about 0.25 wt. % to about 4 wt. %, and about 0.5 wt. % to about 3 wt. %.

An advantage of the skin disinfectant composition that has been observed is the release of chlorhexidine as a result of hand washing. That is, after a person has applied the skin disinfectant composition to his or her hands, a subsequent rinsing or washing of the hands tends to release chlorhexidine so that the chlorhexidine is available to provide disinfectant properties on the skin tissue. The release of chlorhexidine as result of washing of hands can be observed during the useful life of the skin disinfectant composition after application to the hands.

Depending upon the selection of the acid used to form the chlorhexidine salt, the chlorhexidine salt can be more soluable in the polymer phase or more soluable in the water phase. In addition, multiple acids can be selected to provide a split between the chlorhexidine salt where some of the chlorhexidine salt is more soluble than the polymer phase and some of the chlorhexidine salt is more soluble in the water phase. It is generally understood that the chlorhexidine salt provided in the water phase will be available relatively immediately upon application of the skin disinfectant composition to skin tissue to provide disinfectant properties. The chlorhexidine salt that is soluble in the polymer phase is expected to provide more durable or long lasting disinfectant properties.

### Water

The disinfectant active ingredient containing composition can include water in an amount sufficient to allow the composition to be applied to skin tissue while providing the desired coverage over the skin tissue. The water component can be provided as deionized water, filtered water, distilled water, reverse osmosis water, or tap water. In the event that the water includes hardness or other components, it may be desirable to include builders, sequestrants, and chelating agents to handle the water hardness. In general, the composition can include at least about 50 wt. % water. In addition, it is expected that if there is too much water, the emulsion might become unstable. In general, the amount of water in the composition can be up to about 98 wt. %. The amount of water in the composition can be about 65 wt. % to about 97 wt. %, and about 70 wt. % to about 95 wt. %.

### Long Chain Organic Acid

The disinfectant active ingredient containing composition can include a long carbon chain containing organic acid. A long carbon chain containing organic acid can be characterized as an organic acid having a carbon chain that is sufficiently long to help compatibilize or contain the disinfectant active ingredient with the polymer component. While not being bound by theory, it is generally understood that the polymer component in the hydrated composition forms relatively small micelles having a hydrophobic exterior. The long carbon chain on the organic acid compatibilizes with the hydrophobic exterior of the micelles formed by the polymer component, and the acid portion can attract or hold onto the disinfectant active ingredient by, for example, ionic bonding. The long carbon chain containing organic acid can be referred to as the long chain organic acid or even more simply as the organic acid.

If the carbon chain on the long carbon chain organic acid is too short, it may be difficult for the long chain organic acid to interact or combine with the polymer component or adduct. For example, the long chain organic acid can have a carbon chain that is at least about 8 carbon atoms long, and can have a carbon chain that is at least about 10 carbon atoms long. If the carbon chain on the long chain organic acid is too long, it may be difficult to process the organic acid. For example, the long chain organic acid can have a carbon chain that is less than about 22 carbon atoms long, and can have a carbon chain that is less that about 20 carbon atoms long. The long chain organic acid can have a carbon chain of about 10 carbon atoms to about 18 carbon atoms. In addition, the long chain organic acid can have a carbon chain of about 12 carbon atoms to about 16 carbon atoms. Exemplary long chain organic acids that can be used include lauric acid, stearic acid, palmitic acid, myristic acid, and mixtures thereof.

The long chain organic acid can be provided as a component having a single acidic group or multiple acidic groups. Exemplary long chain organic acids that contain multiple acidic groups include those characterized as di-acids and tri-acids. Bonding or other interaction can occur between each acidic group of the long chain organic acid and molecules of disinfectant active ingredients.

The composition can contain the long chain organic acid in an amount sufficient to assist with the stabilization or controlled release of the cationic active ingredient. The amount of the long chain organic acid can be selected depending upon the amount of the cationic active ingredient provided in the composition. If there is a very small amount of the cationic active ingredient, it may be possible to use a relatively small amount of the long chain organic acid in order to compatibilize the cationic active within the composition. For example, the long chain organic acid can be provided in the composition in an amount of at least about 0.05 wt. %. In addition, the amount of the long chain organic acid in the composition can be provided in an amount of at least about 0.5 wt. %. By way of example, the composition can contain about 0.5 wt. % to about 10 wt. % of the long chain organic acid, and can contain about 1.0 wt. % to about 8 wt. % of the long chain organic acid.

### pH Adjusting Agent

The composition can include pH adjusting agents, buffering agents, or neutralizing agents to provide the composition with a pH that helps stabilize the disinfectant active ingredient. Exemplary pH adjusting agents that can be used include sodium hydroxide, potassium hydroxide, triethanolamine, acetic acid, propionic acid, citric acid, succinic acid, gluconic acid, and mixtures thereof.

The polymer component of the lotion, cream, gel, or liquid may be at least in part responsible for reducing the irritability of the composition. For example, it is believed that the polymer component may help reduce irritation of skin tissue. The composition can be provided without any pH modifier, if desired. In general, however, a buffering agent is incorporated into the composition to help control the pH of the composition. Furthermore, the buffering agent is typically selected as a buffering agent that is compatible with skin issue or that does not harm skin tissue. The composition can be provided having an exemplary pH of about 3 to about 6, and can have an exemplary pH of about 3.5 to about 5.

### Release Agent

The disinfectant active ingredient containing composition can include a release agent to assist with the sustained release of the disinfectant active component over a prolonged period of time. The release agent can be provided as a surfactant. A surfactant can additionally be present to help maintain the disinfectant active ingredient containing composition as an emulsion. In general, an emulsion refers to a composition that resists phase separation after sitting at room temperature for a couple of months. In general, it is expected that the disinfectant active ingredient containing composition can be stored in a warehouse or in a storage closet for at least two months and can remain as an emulsion during that two month period. Preferably, the composition can remain as an emulsion for at least one year or at least two years. The ability of the disinfectant active ingredient containing composition to remain as an emulsion can be tested according to an accelerated stability test where the composition is held at 40° C. for 120 days. It is expected that this accelerated stability test for 120 days roughly corresponds to a period of about two years at room temperature. In general, it is expected that the composition can remain as an emulsion after sitting for two years at room temperature.

Exemplary surfactants that can be used as the surfactant component include nonionic surfactants that help stabilize the emulsion and provide a generally even distribution of the disinfectant active ingredient containing component. Exemplary nonionic surfactants that can be used include glycerol stearate such as glycerol monostearate, polysorbate such as that available under the name Tween 80, polyoxyethylene stearate. In addition, mixtures of nonionic surfactants can be included including mixtures of polysorbate and glycerol stearate. An additional nonionic surfactant that can be used includes an ethoxy surfactant, a propoxy surfactant, or an ethoxy/propoxy surfactant. An exemplary ethoxy/propoxy surfactant includes a 10 carbon chain and 9 PO/EO surfactant available under the name Lutensol XP-90 from BASF. Additional nonionic surfactants include sorbitan monolaurate and sorbitan monostearate. Additional surfactants that can be used include those that are generally characterized as Pluronic surfactants such as poloxamers. An exemplary surfactant that can be used is Pluronic L44NF from BASF.

It is believed that anionic surfactants may be useful as part of the surfactant component. In general, it is expected that anionic surfactants have a greater tendency to cause irritation to skin tissue.

The disinfectant active ingredient containing composition can include an amount of surfactant component sufficient to provide the composition with a desired emulsion stability and sufficiently low viscosity without foaming. The amount of the surfactant component in the composition, can be about 0.5 wt. % to about 6 wt. %, and can be about 1 wt. % to about 5 wt. %. It should be understood that the composition can be provided without any surfactant component, if desired.

The composition can contain a release agent to assist with the sustained release of the disinfectant active ingredient over a prolonged period of time. A sustained release of the disinfectant active ingredient refers to a release, over the time period, wherein the release provides desired properties. In general, it is desirable for the disinfectant active ingredient containing composition to provide a relatively consistent release of the disinfectant active ingredient component after application of the composition to skin tissue. A relatively consistent release can be characterized as a release rate at one hour that is within about 50% of the release rate at 30 minutes. In addition, a relatively consistent release rate can be characterized as a release rate at two hours that is within about 50% of the release rate at 30 minutes. Preferably, these release rates can be provided within about 25%, and more preferably can be provided within about 15%.

At least two advantages can be obtained by providing a sustained release rate or a relatively constant release rate over a prolonged period of time. For example, by providing a sustained release of the disinfectant active ingredient over a prolonged period of time, it is possible to prolong the pharmaceutical efficacy of the disinfectant active ingredient containing composition after application to skin tissue. By prolonging the pharmaceutical efficacy of the disinfectant active ingredient containing composition, it is expected that enhanced performance can be achieved. Furthermore, by controlling the release of the disinfectant active ingredient so that it is not released at one instant in time, it is possible to reduce or minimize skin irritation. Many disinfectant active ingredients have a tendency to cause skin irritation if provided at a concentration that is too high. By controlling the release of the disinfectant active ingredient, it is possible to reduce the tendency of the disinfectant active ingredient to cause skin irritation because too much of it is released at one time.

### Thickener

Thickeners that can be incorporated into the composition include those components that thicken or increase the viscosity of the composition so that the composition can be readily applied to skin. Thickeners that can be used in the composition include those components often referred to as viscosity controlling agents.

Exemplary thickeners or viscosity controlling agents that can be provided in the hand disinfecting composition include cellulose gum, alkane triols; acrylates; substituted celluloses such as hydroxy ethyl cellulose, carboxymethyl cellulose, methylcellulose, and hydroxypropyl cellulose; cetyl alcohol; gums such as natural gums or synthetic gums; long chain alcohols such as those having about 9 to about 24 carbon atoms; polyglycols such as polyethylene glycols, polypropylene glycols, polybutylene glycols, polyethylene propylene glycols, or mixtures thereof; waxes such as natural waxes or synthetic waxes; hydrogenated oils; glycol esters; fatty acid esters; long chain acids; acid amides; silicates; and mixtures thereof. Exemplary thickeners that can be used is hydroxyethyl cellulose. An exemplary thickener that can be used is a polyacrylic acid thickeners available under the name Carbopol from Lipscomb.

The thickener is preferably a cationic thickener. An exemplary cationic thickener includes a polyacrylate thickener that can be referred to polyacrylate-1 crosspolymer. An exemplary polyacrylate-1 crosspolymer is available under the name Carbopol Aqua CC from Lipscomb. The cationic thickener available under the name Carbopol Aqua CC is a 20% solution, which means that the composition contains 80 wt. % water.

The composition may or may not include a thickener. When the composition includes a thickener, the thickener can be provided in an amount that provides the desired level of thickening. The composition can include a thickener in an amount of least about 0.1 wt. % and can include a thickener in an amount of at least about 0.4 wt. %. In addition, the thickener can be provided in an amount of less than about 4 wt. %, and can be provided in an amount of less than about 2 wt. %. It should be understood that this weight percent range refers to the dry weight of the thickener. In the situation where the thickener is in solution, the identified weight range refers to the dry weight.

### Emollient

The composition can include an emollient for improving the texture of the composition. An emollient is an oleaginous or oily substance which helps to smooth and soften the skin, and may also reduce its roughness, cracking or irritation. Exemplary suitable emollients include mineral oil, having a viscosity in the range of 50 to 500 centipoise (cps), lanolin oil, coconut oil, cocoa butter, olive oil, almond oil, macadamia nut oil, synthetic jojoba oils, natural sonora jojoba oils, safflower oil, corn oil, liquid lanolin, aloe vera, cottonseed oil, and peanut oil.

Other suitable emollients include squalane, castor oil, polybutene, odorless mineral spirits, sweet almond oil, avocado oil, clophyllum oil, ricin oil, vitamin E acetate, olive oil, linolenic alcohol, coconut oil, oleyl alcohol, the oil of cereal germs such as the oil of wheat germ, isopropyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricin oleates of alcohols and poly alcohols such as those of isopropyl adipate, hexyl laurate and octyl dodecanoate.

Other suitable emollients which are solids or semi-solids at room or ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol and isocetyl lanolate. Exemplary emollients include stearic acid, stearyl alcohol, palmitic acid enters natural and synthetic esters such as coconut oil.

The disinfectant active ingredient containing composition can include the emollient in an amount sufficient to provide a silky feel. An exemplary range of the emollient in the composition can be at least about 0.5 wt. %. In addition, the composition can include an emollient in an amount of less than about 3 wt. %. It should be understood that the emollient is an optional component of the composition. The disinfectant active ingredient containing composition can be provided without an emollient, if desired.

### Moisturizer

The composition can include a moisturizer to provide a desired moisturizing effect to skin tissue. The moisturizer can be provided as a humectant. In general, a humectant is a moistening agent that promotes retention of water due to its hydroscopic properties. Exemplary humectants include glycerine, polymeric glycols such as polyethylene glycol and polypropylene glycol, and sorbitols such as sorbitol solution, pyrrolidone carboxylic acid, urea, or mixtures thereof. The composition can be provided without a moisturizer.

When the disinfectant active ingredient composition includes a moisturizer, it can be included in an amount of at least about 0.5 wt. %. In addition, the composition can include a moisturizer in an amount of less than about 5 wt. %.

An additional component that can be provided as part of the composition is a skin protectant. An exemplary protectant is allantoin that is available as a skin protectant and esthetic agent.

### Preservatives

The composition can include preservatives for prevention of bacterial, fungal, and/or yeast contamination. Exemplary preservatives that can be used in the hand disinfecting composition include phenoxyethanol, benzoic acid, derivatives and salts of benzoic acid, parabens, oxazolidines, chlorinated aromatic compounds and phenols, hydantoins, cresols and derivatives, imiazolindinyl urea, iodopropanol butylcarbamate, sulfites, and bisulfites. The composition can include any of the preservatives commonly used or known to be suitable for topically applied compositions. Exemplary commercially available preservatives include liquid Germal Plus (diazolidinyl urea and iodopropynyl butylcarbamate) and Germaben 11 (diazolidinyl urea and methylparaben and propylparaben).

The composition can be formulated without a preservative. It is expected that the preservative will increase the shelf life of the composition by reducing or preventing the growth of bacteria, fungus, and/or yeast. When the composition includes a preservative, the preservative is preferably provided in an amount sufficient to provide a desired level of protection from growth of bacteria, fungus, and/or yeast.

In general, for most preservatives, it is expected that the amount of preservative can be provided at a level of about 0.1 wt. % to about 1.0 wt. %, and can be provided at a level of about 0.2 wt. % to about 0.5 wt. %, based on the weight of the composition.

### Antioxidants

The composition can include antioxidants to help increase the shelf life of the composition and to provide desired properties when applied to skin tissue. Exemplary antioxidants that can be used include vitamins such as vitamin E, vitamin E acetate, vitamin C, and vitamin D, and derivatives thereof. Exemplary antioxidants include α-tocopherols which can be characterized as natural or synthetic Vitamin E. Additional exemplary antioxidants include propyl, octyl and dodecyl esters of gallic acid, butylated hydroxyanisole (BHA)(usually as a mixture of ortho and meta isomers), butylated hydroxytoluene (BHT), and nordihydroguaiaretic acid, and alkylated parabens such as methylparaben and propylparaben.

The composition can be formulated without an antioxidant. When the composition includes an antioxidant, the antioxidant can be provided in an amount that provides antioxidant properties in the composition. In general, it is expect that the antioxidant can be provided in an amount of about 0.2 wt. % to about 2 wt. %, and can be provided in an amount of about 0.7 wt. % to about 1.5 wt. %, based on the weight of the composition. In the case of vitamin E, it is expected that the vitamin E can be included in the composition in an amount of about 0.1 wt. % to about 1 wt. %, and can be included in an amount of about 0.3 wt. % to about 0.8 wt. %.

### Chelating Agents

Chelating agents are substances used to chelate or bind metallic ions with a certain heterocyclic ring structure so that the ion is held by chemical bonds from each of the participating rings. Suitable chelating agents include ethylene diaminetetraacetic acid (EDTA), EDTA trisodium, EDTA tetrasodium, calcium disodium edetate, EDTA trisodium, EDTA tetrasodium and EDTA dipotassium. One or more chelating agents can optionally be included in the emulsion in amounts ranging from about 0.001 to about 0.1 weight percent. It should be appreciated that the composition can be provided without a chelating agent.

### Fragrances

Fragrances are aromatic compounds which can impart an aesthetically pleasing aroma to the composition. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the composition in an amount ranging from about 0.001 to about 10 weight percent, preferably about 0.05 to about 5 percent. It should be appreciated that the composition can be provided without a fragrance.

### Carriers, Diluents, and Excipients

The composition may also include non-toxic, pharmaceutically and dermatologically acceptable carriers, diluents and excipients, suitable for topical application, as are well known, see for example Merck Index, Merck & Co., Rahway, N.J., Bioreversible Carriers in Drug Design, Theory and Application, Roche (ed.) Pergamon Press, (1987), Gilman et al., (eds) (1990) Goodman and Gilman's: The Pharmacological Bases of Therapeutics, 8th Ed., Pergamon Press, Novel Drug Delivery Systems, 2nd Ed., Norris (ed.) Marcel Dekker Inc., (1989), and Remington's Pharmaceutical Sciences. For standard dosages of conventional pharmacological agents, see, e.g., Physicians Desk Reference (1997 Edition); and American Medical Association (1997) Drug Evaluations (Subscriptions).

The disinfectant active ingredient containing composition can be applied to skin tissue as a result of applying the composition as a cream or lotion or gel or liquid to the skin tissue, and rubbing onto the skin tissue. The action of rubbing may include gentle rubbing or vigorous rubbing. Although the composition is sometimes characterized as having a binding or almost adhesive property with respect to skin tissue, the composition is not the type of composition that one would consider to be an adhesive that holds two substrates together such as, for example, a hot melt adhesive.

Because the disinfectant active ingredient containing composition can bind cationic active ingredients therein, the release of the active ingredient can take place over time. In contrast, many currently available compositions release the active ingredient almost instantaneously. As a result, the dose of the active ingredient from these compositions that release almost instantaneously may be too great at one time and too low soon thereafter. Furthermore, the release of a high dosage may cause skin irritation. By providing a prolonged release of the active ingredient over a period of time, the efficacy of the drug can be improved. The ability of the composition to bind the cationic active ingredient and provide a gradual or sustained release over time can be demonstrated by a two hour release from a nylon membrane, property as described in Example 1. The disinfectant active ingredient containing composition can provide a two hour release from a nylon membrane property of less than 80%. If desired, the composition can provide a two hour release from nylon membrane property of less than 50%. It is expected that a composition that does not bind the active ingredient will provide a two hour release from a nylon membrane property of greater than 90%.

### Example

A skin disinfectant composition was prepared from the components identified in Table 1.

| Component | Wt. % |
|---|---|
| Water | 76.85 |
| Cationic thickener (Carbopol Aqua CC) | 5.00 |
| Chlorhexidine base | 2.25 |
| Compatibilizer (stearic acid) | 3.00 |
| Buffering agent (succinic acid) | 0.60 |
| Polymer adduct | 6.00 |
| Emollient (coconut oil) | 5.00 |
| pH neutralizer (gluconic acid) | 0.80 |
| Nonionic surfactant (Pluronic L44 NF) | 0.50 |

The cationic thickener can be referred to as polyacrylate-1 crosspolymer, and is available as a cationic polyacrylate thickener as a 20% solution in water. That means that the composition contains 80 wt. % water and 20 wt. % of the polyacrylate thickener. The chlorhexidine base contains 4% chlorhexidene gluconate which can act as a solubilizer and prevent precipitate from forming. The compatibilizer can be stearic acid and helps compatibilize the chlorhexidine base with the polymer adduct. The buffering agent is provided to maintain a pH for the composition of about 4.5. The pH neutralizer can be gluconic acid and is provided as a 50% solution. The gluconic acid helps solubilize some of the chlorhexidine, and is believed to help prevent the succinic acid from reacting with chlorhexidine and forming an insoluble precipitate. The polymer adduct is formed from the following components:

| | |
|---|---|
| Polyquaternium 44 (20% solution) | 64 wt. % |
| Polyvinylpyrrolidone/hexaderene | 25 wt. % |
| Polyvinylpyrrolidone/licosene | 5 wt. % |
| Polyvinylpyrrolidone/tricontanyl | 5 wt. % |
| Preservative | 1 wt. % |

The skin disinfectant composition is prepared by mixing the cationic thickener in water at 65° C. to form a water phase. The stearic acid, coconut oil, surfactant, and polymer adduct are added together and mixed at 65° C. The chlorhexadine base is then added. Succinic acid and gluconic acid are then added. Finally, the water phase is added and the composition is mixed and allowed to cool.

The polymer adduct is formed by mixing the components of the polymer adduct at a temperature of about 60° C., and then allowing the composition to cool to room temperature.

The composition reported in Table 1 can be referred to as composition A. The skin disinfectant properties of Composition A were compared with the skin disinfectant properties of Composition B and Composition C. Composition B is a hand sanitizer sold under the name Purell®. Composition B contains about 62 wt. % alcohol. Composition C is a product available under the name HIBICLENS from Regent Medical. The HIBICLENS product is aqueous and contain 4.0 wt. % chlorhexidine gluconate and 4.0 wt. % isopropanol. For purposes of this test, the Purell® and HIBICLENS products were used according to their instructions. The Purell® product was applied the hand and rubbed onto the skin tissue. The HIBICLENS product was used as a soap product. Composition A was applied to the hands and rubbed in a manner similar to the Purell® product.

The following test was carried out by three individuals on five consecutive days. For each day, the individuals were told not to wash their hands for at least two hours before the test. The same three people were the subjects of the tests on the five consecutive days. On each day, the subjects would touch an agar plate with their hand. This initial touch is before application of the compositions A-C. The composition was then applied to the hands according to the instructions on the product. On day 1, the subjects applied Composition A to their hands. On day 2, the subjects applied Composition B to their hands. On day 3, the subjects applied Composition C to their hands. On days 4 and 5, the subjects applied Composition A to their hands. After application of the composition to the hands, and after the hands were allowed to dry, the subjects were told to touch another agar plate. After each of one hour, two hours, three hours, four hours, the subjects were told to touch an agar plate. The agar plates were collected and incubated at 35° C. for three days. The plates were removed and the CFUs (Colony Forming Units) for each plate were counted. The average results are reported in Table 2.

Composition A with rinse means that before the one hour, two hour, three hour, four hour tests, the subjects hands were rinsed for 30 seconds in water and allowed to dry. The Composition A with wash means that before the tests at one hour, two hours, three hours, and four hours, the subjects were instructed to wash their hand with mild soap for 30 seconds and allows their hands to dry. After the rinsing or washing steps, the subjects were asked to wait 10 minutes before touching the agar plate.

TABLE 1
CFU Comparison

| | Pre Apply | Apply | 1 hr | 2 hr | 3 hr | |
|---|---|---|---|---|---|---|
| | CFU | CFU | CFU | CFU | CFU | 4 hr CFU |
| Composition A | 47 | 0 | 0 | 0 | 1 | 2 |
| Composition B | TNTC* | 2 | 12 | 85 | 34 | 32 |
| Composition C | 3 | 2 | 11 | 30 | 159 | 94 |
| Composition A* | TNTC* | 0 | 0 | 0 | 0 | 0 |
| rinse | | | | | | |
| Composition A* | 29 | 0 | 0 | 0 | 0 | 2 |
| Wash | | | | | | |

*Too numerous to count.

Based on the data reported in Table 2, Composition A significantly out preformed Composition B and C.

The above specification, examples and data provide a complete description of the manufacture and use of the composition of the invention. Since many embodiments of the invention can be made without departing from the spirit and scope of the invention, the invention resides in the claims hereinafter appended.

We claim:

1. A disinfectant active ingredient containing composition comprising:
(a) about 0.1 wt. % to about 6 wt. % disinfectant active ingredient;
(b) at least about 2 wt. % of a hydrophobic polymer/hydrophilic polymer adduct comprising a poly(vinylpyrrolidone-alkylene) copolymer wherein the alkylene group contains at least 10 carbon atoms and a poly(vinylpyrrolidone imidizole) copolymer;
(c) a compatibilizing amount of a long chain organic acid having a carbon chain of at least about 8 carbon atoms; and
(d) at least about 50 wt. % water.

2. A disinfectant active ingredient containing composition according to claim 1, wherein the poly(vinlypyrrolidone-alkylene) copolymer comprises a polymer having an alkylene group containing about 10 carbon atoms to about 32 carbon atoms.

3. A disinfectant active ingredient containing composition according to claim 1, wherein the poly(vinylpyrrolidone-alkylene) copolymer comprises a mixture of poly(vinylpyrrolidone-alkylene) copolymer wherein the alkylene group contains 10 carbon atoms to 24 carbon atoms and poly(vinylpyrrolidone-alkylene) polymer wherein the alkylene group contains 26 carbon atoms to 32 carbon atoms.

4. A disinfectant active ingredient containing composition according to claim 1, wherein the poly(vinylpyrrolidone imidizole) copolymer comprises polyvinylpyrrolidone vinylimidizolemethylsulfonate copolymer.

5. A disinfectant active ingredient containing composition according to claim 1, wherein the weight ratio of the poly(vinylpyrrolidone-alkylene) copolymer wherein the alkylene group contains at least 10 carbon atoms to the poly(vinylpyrrolidone imidizole) copolymer is about 6:1 to about 1:1.

6. A disinfectant active ingredient containing composition according to claim 1, wherein the long chain organic acid has a carbon chain of about 10 carbon atoms to about 20 carbon atoms.

7. A disinfectant active ingredient containing composition according to claim 1, wherein the long chain organic acid has a carbon chain of about 12 carbon atoms to about 16 carbon atoms.

8. A disinfectant active ingredient containing composition according to claim 1, wherein the long chain organic acid comprises at least one of lauric acid, stearic acid, palmitic acid, myristic acid, or a mixture thereof.

9. A disinfectant active ingredient containing composition according to claim 1, wherein the composition comprises at least about 0.05 wt. % of the long chain organic acid.

10. A disinfectant active ingredient containing composition according to claim 1, wherein the composition comprises about 0.5 wt. % to about 6 wt. % of a surfactant.

11. A disinfectant active ingredient containing composition according to claim 1, wherein the composition comprises about 0.1 wt. % to about 2 wt. % of a thickener.

12. A disinfectant active ingredient containing composition according to claim 1, wherein the composition comprises about 0.5 wt. % to about 5 wt. % of a moisturizer.

13. A disinfectant active ingredient containing composition according to claim 1, wherein the composition has a pH of about 3.5 to about 7.

14. A disinfectant active ingredient containing composition according to claim 1, wherein the two hour release from a nylon membrane property of the disinfectant active ingredient is less than 80%.

15. A method of using a disinfectant active ingredient containing composition, the method comprises:
(a) applying a disinfectant active ingredient containing composition to skin tissue, the disinfectant active ingredient containing composition comprising:
(i) about 0.1 wt. % to about 6 wt. % disinfectant active ingredient;
(ii) at least about 2 wt. % of a hydrophobic polymer/hydrophilic polymer adduct comprising a poly(vinylpyrrolidone-alkylene) copolymer wherein the alkylene group contains at least 10 carbon atoms and a poly(vinylpyrrolidone imidizole) copolymer;
(iii) a compatibilizing amount of a long chain organic acid having a carbon chain of at least about 8 carbon atoms; and
(iv) at least about 50 wt. % water.

16. A method according to claim 15, wherein the poly(vinlypyrrolidone-alkylene) copolymer comprises a polymer having an alkylene group containing about 10 carbon atoms to about 30 carbon atoms.

17. A disinfectant active ingredient containing composition according to claim 15, wherein the poly(vinylpyrrolidone-alkylene) copolymer comprises a mixture of poly(vinylpyrrolidone-alkylene) copolymer wherein the alkylene group contains 10 carbon atoms to 24 carbon atoms and poly(vinylpyrrolidone-alkylene) copolymer wherein the alkylene group contains 26 carbon atoms to 32 carbon atoms.

18. A disinfectant active ingredient containing composition according to claim 15, wherein the poly(vinylpyrrolidone imidizole) copolymer comprises polyvinylpyrrolidone vinylimidizolemethylsulfonate copolymer.

19. A disinfectant active ingredient containing composition according to claim 15, wherein the weight ratio of the poly(vinylpyrrolidone-alkylene) copolymer wherein the alkylene group contains at least 10 carbon atoms to the poly(vinylpyrrolidone imidizole) copolymer is about 6:1 to about 1:1.

20. A method according to claim 15, wherein the composition comprises about 0.5 wt. % to about 6 wt. % of a surfactant.

21. A method according to claim 15, wherein the composition comprises about 0.1 wt. % to about 2 wt. % of a thickener.

22. A method according to claim 15, wherein the composition comprises about 0.5 wt. % to about 5 wt. % of a moisturizer.

23. A method according to claim 15, wherein the long chain organic acid has a carbon chain of about 10 carbon atoms to about 20 carbon atoms.

24. A method according to claim 15, wherein the long chain organic acid has a carbon chain of about 12 carbon atoms to about 16 carbon atoms.

25. A method according to claim 15, wherein the long chain organic acid comprises at least one of lauric acid, stearic acid, palmitic acid, myristic acid, or a mixture thereof.

26. A method according to claim 15, wherein the composition comprises at least about 0.05 wt. % of the long chain organic acid.

27. A method according to claim 15, wherein the two hour release from a nylon membrane property for the disinfectant active ingredient is less than 80%.

28. A method for manufacturing a disinfectant active ingredient containing composition, the method comprising:
(a) forming a polymer adduct comprising a poly(vinylpyrrolidone-alkylene) copolymer wherein the alkylene group contains at least 10 carbon atoms and a poly(vinylporrolidone imidizole) copolymer; and
(b) forming an emulsion containing the polymer adduct and water, wherein the water is present in an amount of at least about 50 wt. %.

29. A disinfectant active ingredient containing composition comprising:
(a) about 0.1 wt. % to about 6 wt. % disinfectant active ingredient;
(b) at least about 2 wt. % of a hydrophobic polymer/hydrophilic polymer adduct comprising a poly(vinylpyrrolidone-alkylene) copolymer wherein the alkylene group contains at least 10 carbon atoms and a poly(vinylpyrrolidone imidizole) copolymer; and
(c) at least about 50 wt. % water.
